(19) United States
(12) Patent Application Publication
(10) Pub. No.: US 2009/0297584 A1
(43) Pub. Date: Dec. 3, 2009

(54) BIOSOLUBLE COATING WITH LINEAR OVER TIME MASS LOSS

(76) Inventors: FLORENCIA LIM, Union City, CA (US); Michael H. Ngo, San Jose, CA (US); Mikael O. Trollsas, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 12/195,325

(22) Filed: Aug. 20, 2008

Related U.S. Application Data
(63) Continuation-in-part of application No. 12/106,212, filed on Apr. 18, 2008.

(51) Int. Cl. A61K 9/00

(52) U.S. Cl. 424/426

(57) ABSTRACT

Implantable devices such as stents having a biosoluble coating with linear over time mass loss are provided. Also disclosed are methods of making and using the implantable device.

### CROSS-REFERENCE TO RELATED APPLICATION

This application is a continuation-in-part of U.S. application Ser. No. 12/106,212, filed Apr. 18, 2008, the teaching of which is incorporated herein in its entirety by reference.

### BACKGROUND OF THE INVENTION

1. Field of the Invention

This invention generally relates to a biosoluble coating with a linear over time mass loss rate in vivo.

2. Description of the Background

Percutaneous coronary intervention (PCI) is a procedure for treating heart disease. A catheter assembly having a balloon portion is introduced percutaneously into the cardiovascular system of a patient via the radial, brachial or femoral artery. The catheter assembly is advanced through the coronary vasculature until the balloon portion is positioned across the occlusive lesion. Once in position across the lesion, the balloon is inflated to a predetermined size to radially compress the atherosclerotic plaque of the lesion to remodel the lumen wall. The balloon is then deflated to a smaller profile to allow the catheter to be withdrawn from the patient's vasculature.

Problems associated with the above procedure include formation of intimal flaps or torn arterial linings which can collapse and occlude the blood conduit after the balloon is deflated. Moreover, thrombosis and restenosis of the artery may develop over several months after the procedure, which may require another angioplasty procedure or a surgical by-pass operation. To reduce the partial or total occlusion of the artery by the collapse of the arterial lining and to reduce the chance of thrombosis or restenosis, a stent is implanted in the artery to keep the artery open.

Drug delivery stents have reduced the incidence of in-stent restenosis (ISR) after PCI (see, e.g., Serruys, P. W., et al., J. Am. Coll. Cardiol. 39:393-399 (2002)), which has plagued interventional cardiology for more than a decade. However, a few challenges remain in the art of drug delivery stents. For example, release of a drug from a coating formed of a bulk-eroding polymer often have a burst release of the drug, resulting in insufficient control release of the drug.

Therefore, there is a need for a coating that provides for a controlled release of a drug in the coating.

The embodiments of the present invention address the above-identified needs and issues.

### SUMMARY OF THE INVENTION

In according to one aspect of the present invention, it is provided an implantable device comprising a copolymer, which is biosoluble, and upon exposure to a physiological environment, 80% mass of the copolymer will dissolve in a period of about 1 day to about 30 days and has a linear over time mass loss. Examples of such copolymers include poly(lactic acid-co-glycolic acid)-poly(ethylene glycol) (PLGA-PEG) block copolymer, other PEG copolymers, PEG, polyvinyl alcohol (PVA), poly(vinyl pyrrolidone) (PVP), hyaluronic acid, hydroxyl cellulose, polysaccharides, phosphoryl choline polymers, and other hydrophilic polymers.

In some embodiments, the implantable device comprises a block copolymer that comprises at least one polyester block and at least one poly(ethylene glycol) (PEG) block. The PEG block has a weight average molecular weight (M_{w}) from about 1,000 Daltons to about 30,000 Daltons. The polyester block(s) in the block copolymer comprises glycolide, lactide, trimethylene carbonate, caprolactone, or combinations thereof. The lactide can be optically active or racemic and can be D,L-lactide, L-lactide, D-lactide, or combinations thereof. The polyester block(s) can have various molar concentrations of any of these monomers. For example, the polyester block(s) can have lactide with a molar concentration in the polyester block(s) of at least 60% or at least 80%. In such embodiments, the polyester block(s) can further have glycolide with a molar concentration in the polyester block(s) of between about 10% and about 75%. An example of such polymers are PLGA-PEG-PLGA block copolymer with PEG ranging from about 5 mol % to about 50 mol %, more specifically 15 mol % to about 30 mol % (e.g., 17 mol % or 22 mol %). In this polymer, the lactide monomer can have different molar ratio to the glycolide monomer, ranging from e.g., about 10:90 to about 90:10, e.g., about 20:80, about 30:70, about 40:60, about 50:50, about 60:40, about 70:30, or about 80:20.

In some embodiments, the polymer or copolymer can comprise biodegradable side blocks. The side blocks can be any biodegradable polymer, a few examples of which are polyanhydrides, poly(ester amides), polythioesters, or combinations thereof.

In some embodiments, the polymer or copolymer can be an alternating A-B block copolymer where A is a poly(lactide-co-glycolide) (PLGA) block and B is the PEG block.

A few non-limiting examples of the block copolymer are poly(lactide-co-glycolide-co-caprolactone)-block-PEG-poly(lactide-co-glycolide-co-caprolactone), poly(trimethylene carbonate-co-glycolide)-block-PEG-block-poly(trimethylene carbonate-co-glycolide), polylactide-block-PEG-polyactide, poly(trimethylene carbonate-co-glycolide)-block-PEG-poly(trimethylene carbonate-co-glycolide), and combinations thereof.

The polymer or copolymer of the various embodiments above can form a coating on the implantable device or at least a portion of the body structure of the implantable device. In some embodiments, the coating or the body structure of the implantable device can further comprise a bioactive agent. Some examples of the bioactive agent can be paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl(4-amino-TEMPO), biolimus, tacrolimus, dexamethasone, dexamethasone acetate, corticosteroids, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), zotarolimus, Biolimus A9 (Biosensors International, Singapore), AP23572 (Ariad Pharmaceuticals), γ-hiridun, clobetasol, pimecrolimus, imatinib mesylate, midostaurin, cRGD, feno fibrate, peptides, prodrugs thereof, co-drugs thereof, and combinations thereof.

The implantable device can be any implantable device such as a stent. The implantable device can be biodurable or bioabsorbable. In some embodiments, the implantable device is a bioabsorbable stent.

In according to a further aspect of the present invention, it is provided a method of fabricating an implantable medical device. The method comprises forming a biosoluble coating on the implantable device of the various embodiments described above.

The implantable device described herein can be formed on an implantable device such as a stent, which can be implanted in a patient to treat, prevent, mitigate, or reduce a vascular medical condition, or to provide a pro-healing effect.

In some embodiments, the vascular medical condition or vascular condition is a coronary artery disease (CAD) and/or a peripheral vascular disease (PVD). Some examples of such vascular medical diseases are restenosis and/or atherosclerosis.

Some other examples of these conditions include thrombosis, hemorrhage, vascular dissection or perforation, vascular aneurysm, vulnerable plaque, chronic total occlusion, claudication, anastomotic proliferation (for vein and artificial grafts), bile duct obstruction, urethral obstruction, tumor obstruction, or combinations of these.

### BRIEF DESCRIPTION OF THE DRAWING

FIG. 1 shows linear over time mass loss of an embodiment of the invention biosoluble coating.

### DETAILED DESCRIPTION OF THE INVENTION

In according to one aspect of the present invention, it is provided an implantable device comprising a polymer or copolymer, which is biosoluble and upon, exposure to a physiological environment, 80 mass % of the polymer or copolymer will dissolve in a period of about 1 day to about 30 days and has a linear over time mass loss (LOTML). Examples of such copolymers include poly(lactic acid-co-glycolic acid)-poly(ethylene glycol) (PLGA-PEG) block copolymer, other PEG copolymers, PEG, polyvinyl alcohol (PVA), poly(vinyl pyrrolidone) (PVP), hyaluronic acid, hydroxyl cellulose, polysaccharides, phosphoryl choline polymers, and other hydrophilic polymers.

As used herein, the term “biosoluble” shall also encompass meaning of the term “bioabsorbable” or “biodegradable” in that a bioabsorbable or biodegradable polymer or copolymer, upon exposure to a physiological environment, will absorb or degrade with linear over time mass loss at a rate that 80 mass % or more of the polymer or copolymer will absorb or degrade in a period of about 1 day to about 30 days and.

The implantable device can be any implantable device such as a stent. The implantable device can be biodurable or bioabsorbable. In some embodiments, the implantable device is a bioabsorbable stent.

In some embodiments, the implantable the biosoluble coating comprises a block copolymer comprising at least one polyester block and at least one poly(ethylene glycol) (PEG) block. The PEG block has a weight average molecular weight (M_{w}) from about 1,000 Daltons to about 30,000 Daltons. The polyester block(s) in the block copolymer comprises glycolide, lactide, trimethylene carbonate, caprolactone, or combinations thereof. The lactide can be optically active or racemic and can be D,L-lactide, L-lactide, D-lactide, or combinations thereof. The polyester block(s) can have various molar concentrations of any of these monomers. For example, the polyester block(s) can have lactide with a molar concentration in the polyester block(s) of at least 60% or at least 80%. In such embodiments, the polyester block(s) can further have glycolide with a molar concentration in the polyester block(s) of between about 10% and about 75%. An example of such polymers are PLGA-PEG-PLGA block copolymer with PEG ranging from about 5 mol % to about 50 mol %, more specifically 15 mol % to about 30 mol % (e.g., 17 mol % or 22 mol %). In this polymer, the lactide monomer can have different molar ratio to the glycolide monomer, ranging from e.g., about 10:90 to about 90:10, e.g., about 20:80, about 30:70, about 40:60, about 50:50, about 60:40, about 70:30, or about 80:20. A few other non-limiting examples of the block copolymer are poly(lactide-co-glycolide-co-caprolactone)-block-PEG-poly(lactide-co-glycolide-co-caprolactone), poly(trimethylene carbonate-co-glycolide)-block-PEG-block-poly(trimethylene carbonate-co-glycolide), polylactide-block-PEG-polyactide, poly(trimethylene carbonate-co-glycolide)-block-PEG-poly(trimethylene carbonate-co-glycolide), and combinations thereof.

In some embodiments, the soluble coating disclosed herein comprises a polymer or copolymer comprising biodegradable side blocks. The side blocks can be any biodegradable polymer, a few examples of which are polyanhydrides, poly(ester amides), polythioesters, or combinations thereof.

In some embodiments, the soluble coating comprises a block copolymer which is an alternating A-B block copolymer where A is a poly(lactide-co-glycolide) (PLGA) block and B is the PEG block.

The soluble coating can be formed on the implantable device or at least a portion of the body structure of the implantable device. In some embodiments, the coating or the body structure of the implantable device can further comprise a bioactive agent. Some exemplary bioactive agents that can be included in the soluble coating are paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl(4-amino-TEMPO), biolimus, tacrolimus, dexamethasone, dexamethasone acetate, corticosteroids, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), zotarolimus, Biolimus A9 (Biosensors International, Singapore), AP23572 (Ariad Pharmaceuticals), γ-hiridun, clobetasol, pimecrolimus, imatinib mesylate, midostaurin, cRGD, feno fibrate, peptides, prodrugs thereof, co-drugs thereof, and combinations thereof. Some other examples of the bioactive agent include siRNA and/or other oligoneucleotides that inhibit endothelial cell migration. Some further examples of the bioactive agent can also be lysophosphatidic acid (LPA) or sphingosine-1-phosphate (S1P). LPA is a “bioactive” phospholipid able to generate growth factor-like activities in a wide variety of normal and malignant cell types. LPA plays an important role in normal physiological processes such as wound healing, and in vascular tone, vascular integrity, or reproduction. As used herein, in some embodiments, the term “drug” and the term “bioactive agent” are used interchangeably.

In according to a further aspect of the present invention, it is provided a method of fabricating an implantable medical device. The method comprises forming a soluble coating on the implantable device, the coating comprising a polymer or copolymer of the various embodiments described above.

The implantable device described herein can be formed on an implantable device such as a stent, which can be implanted in a patient to treat, prevent, mitigate, or reduce a vascular medical condition, or to provide a pro-healing effect. In some embodiments, the vascular medical condition or vascular condition is a coronary artery disease (CAD) and/or a peripheral vascular disease (PVD). Some examples of such vascular medical diseases are restenosis and/or atherosclerosis.

Some other examples of these conditions include thrombosis, hemorrhage, vascular dissection or perforation, vascular aneurysm, vulnerable plaque, chronic total occlusion, claudication, anastomotic proliferation (for vein and artificial grafts), bile duct obstruction, urethral obstruction, tumor obstruction, or combinations of these.

### DEFINITIONS

Wherever applicable, the definitions to some terms used throughout the description of the present invention as provided below shall apply. The terms “biologically degradable” (or “biodegradable”), “biologically erodable” (or “bioerodable”), “biologically absorbable” (or “bioabsorbable”), and “biologically resorbable” (or “bioresorbable”), in reference to polymers and coatings, are used interchangeably and refer to polymers and coatings that are capable of being completely or substantially completely degraded, dissolved, and/or eroded over time when exposed to physiological conditions and can be gradually resorbed, absorbed and/or eliminated by the body, or that can be degraded into fragments that can pass through the kidney membrane of an animal (e.g., a human), e.g., fragments having a molecular weight of about 40,000 Daltons (40 K Daltons) or less. The process of breaking down and eventual absorption and elimination of the polymer or coating can be caused by, e.g., hydrolysis, metabolic processes, oxidation, enzymatic processes, bulk or surface erosion, and the like. Conversely, a “biostable” polymer or coating refers to a durable polymer or coating that is not biodegradable.

Whenever the reference is made to “biologically degradable,” “biologically erodable,” “biologically absorbable,” and “biologically resorbable” stent coatings or polymers forming such stent coatings, it is understood that after the process of degradation, erosion, absorption, and/or resorption has been completed or substantially completed, no coating or substantially little coating will remain on the stent. Whenever the terms “degradable,” “biodegradable,” or “biologically degradable” are used in this application, they are intended to broadly include biologically degradable, biologically erodable, biologically absorbable, and biologically resorbable polymers or coatings.

“Physiological conditions” refer to conditions to which an implant is exposed within the body of an animal (e.g., a human). Physiological conditions include, but are not limited to, “normal” body temperature for that species of animal (approximately 37° C. for a human) and an aqueous environment of physiologic ionic strength, pH and enzymes. In some cases, the body temperature of a particular animal may be above or below what would be considered “normal” body temperature for that species of animal. For example, the body temperature of a human may be above or below approximately 37° C. in certain cases. The scope of the present invention encompasses such cases where the physiological conditions (e.g., body temperature) of an animal are not considered “normal.”

In the context of a blood-contacting implantable device, a “prohealing” drug or agent refers to a drug or agent that has the property that it promotes or enhances re-endothelialization of arterial lumen to promote healing of the vascular tissue.

As used herein, a “co-drug” is a drug that is administered concurrently or sequentially with another drug to achieve a particular pharmacological effect. The effect may be general or specific. The co-drug may exert an effect different from that of the other drug, or it may promote, enhance or potentiate the effect of the other drug.

As used herein, the term “prodrug” refers to an agent rendered less active by a chemical or biological moiety, which metabolizes into or undergoes in vivo hydrolysis to form a drug or an active ingredient thereof. The term “prodrug” can be used interchangeably with terms such as “proagent”, “latentiated drugs”, “bioreversible derivatives”, and “congeners”. N. J. Harper, Drug latentiation, Prog Drug Res., 4: 221-294 (1962); E. B. Roche, Design of Biopharmaceutical Properties through Prodrugs and Analogs, Washington, D.C.: American Pharmaceutical Association (1977); A. A. Sinkula and S. H. Yalkowsky, Rationale for design of biologically reversible drug derivatives: prodrugs, J. Pharm. Sci., 64: 181-210 (1975). Use of the term “prodrug” usually implies a covalent link between a drug and a chemical moiety, though some authors also use it to characterize some forms of salts of the active drug molecule. Although there is no strict universal definition of a prodrug itself, and the definition may vary from author to author, prodrugs can generally be defined as pharmacologically less active chemical derivatives that can be converted in vivo, enzymatically or nonenzymatically, to the active, or more active, drug molecules that exert a therapeutic, prophylactic or diagnostic effect. Sinkula and Yalkowsky, above; V. J. Stella et al., Prodrugs: Do they have advantages in clinical practice?, Drugs, 29: 455-473 (1985).

Unless otherwise specifically defined, the terms “polymer” and “polymeric” refer to compounds that are the product of a polymerization reaction. These terms are inclusive of homopolymers (i.e., polymers obtained by polymerizing one type of monomer by either chain or condensation polymers), copolymers (i.e., polymers obtained by polymerizing two or more different types of monomers by either chain or condensation polymers), condensation polymers (polymers made from condensation polymerization, tri-block copolymers, etc., including random (by either chain or condensation polymers), alternating (by either chain or condensation polymers), block (by either chain or condensation polymers), graft, dendritic, crosslinked and any other variations thereof.

As used herein, the term “implantable” refers to the attribute of being implantable in a mammal (e.g., a human being or patient) that meets the mechanical, physical, chemical, biological, and pharmacological requirements of a device provided by laws and regulations of a governmental agency (e.g., the U.S. FDA) such that the device is safe and effective for use as indicated by the device. As used herein, an “implantable device” may be any suitable substrate that can be implanted in a human or non-human animal. Examples of implantable devices include, but are not limited to, self-expandable stents, balloon-expandable stents, coronary stents, peripheral stents, stent-grafts, catheters, other expandable tubular devices for various bodily lumen or orifices, grafts, vascular grafts, arterio-venous grafts, by-pass grafts, pacemakers and defibrillators, leads and electrodes for the preceding, artificial heart valves, anastomotic clips, arterial closure devices, patent foramen ovale closure devices, cerebrospinal fluid shunts, and particles (e.g., drug-eluting particles, microparticles and nanoparticles). The stents may be intended for any vessel in the body, including neurological, carotid, vein graft, coronary, aortic, renal, iliac, femoral, popliteal vasculature, and urethral passages. An implantable device can be designed for the localized delivery of a therapeutic agent. A medicated implantable device may be constructed in part, e.g., by coating the device with a coating material containing a therapeutic agent. The body of the device may also contain a therapeutic agent.

An implantable device can be fabricated with a coating containing partially or completely a biodegradable/bioabsorbable/ bioerodable polymer, a biostable polymer, or a combination thereof. An implantable device itself can also be fabricated partially or completely from a biodegradable/bioabsorbable/bioerodable polymer, a biostable polymer, or a combination thereof.

As used herein, a material that is described as a layer or a film (e.g., a coating) “disposed over” an indicated substrate (e.g., an implantable device) refers to, e.g., a coating of the material deposited directly or indirectly over at least a portion of the surface of the substrate. Direct depositing means that the coating is applied directly to the exposed surface of the substrate. Indirect depositing means that the coating is applied to an intervening layer that has been deposited directly or indirectly over the substrate. In some embodiments, the term a “layer” or a “film” excludes a film or a layer formed on a non-implantable device.

In the context of a stent, “delivery” refers to introducing and transporting the stent through a bodily lumen to a region, such as a lesion, in a vessel that requires treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen.

### Linear Over Time Mass Loss

Linear over time mass loss (LOTML) of the soluble coating can be achieved by various coating engineering. An example of such coating engineering is to adjust ratio of the hydrophobic component, if present, to the hydrophilic component in the coating. In some embodiments, LOTML can be achieved by physically or chemically cross-linking hydrophilic parts of the soluble coating. Physical cross-linking could be achieved through crystalline domains or by using hydrogen bonding, while chemical crosslinking requires the crosslink degradation to either be the rate determining step or to be very fast.

LOTML imparts significant advantages to a coating. An important advantage is release control of a drug or agent from the polymers that have limited miscibility with the drug and therefore prevents diffusion of the drug through the polymer matrix and as a result controlling the drug release rate is difficult to achieve.

Another advantage of LOTML soluble coatings is that the polymers dissolve into the tissue without degradation, and as a result any inflammatory responses caused by the degradation products can be eliminated.

### Biologically Active Agents

In some embodiments, the implantable device described herein can optionally include at least one biologically active (“bioactive”) agent. The at least one bioactive agent can include any substance capable of exerting a therapeutic, prophylactic or diagnostic effect for a patient.

Examples of suitable bioactive agents include, but are not limited to, synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules that bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. The bioactive agents could be designed, e.g., to inhibit the activity of vascular smooth muscle cells. They could be directed at inhibiting abnormal or inappropriate migration and/or proliferation of smooth muscle cells to inhibit restenosis.

In certain embodiments, optionally in combination with one or more other embodiments described herein, the implantable device can include at least one biologically active agent selected from antiproliferative, antineoplastic, antimitotic, anti-inflammatory, antiplatelet, anticoagulant, antifibrin, antithrombin, antibiotic, antiallergic and antioxidant substances.

An antiproliferative agent can be a natural proteineous agent such as a cytotoxin or a synthetic molecule. Examples of antiproliferative substances include, but are not limited to, actinomycin D or derivatives and analogs thereof (manufactured by Sigma-Aldrich, or COSMEGEN available from Merck) (synonyms of actinomycin D include dactinomycin, actinomycin IV, actinomycin II, actinomycin X₁, and actinomycin C₁); all taxoids such as taxols, docetaxel, and paclitaxel and derivatives thereof; all olimus drugs such as macrolide antibiotics, rapamycin, everolimus, structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof. Examples of rapamycin derivatives include, but are not limited to, 40-O-(2-hydroxy)ethyl-rapamycin (trade name everolimus from Novartis), 40-O-(2-ethoxy)ethyl-rapamycin (biolimus), 40-O-(3-hydroxy)propyl-rapamycin, 40-O-[2-(2-hydroxy)ethoxy]ethyl-rapamycin, 40-O-tetrazole-rapamycin, 40-epi-(N1-tetrazolyl)-rapamycin (zotarolimus, manufactured by Abbott Labs.), Biolimus A9 (Biosensors International, Singapore), AP23572 (Ariad Pharmaceuticals), prodrugs thereof, co-drugs thereof, and combinations thereof.

An anti-inflammatory drug can be a steroidal anti-inflammatory drug, a nonsteroidal anti-inflammatory drug (NSAID), or a combination thereof. Examples of anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol, clobetasol propionate, clobetasone butyrate, clopirac, cloticasone propionate, cormethasone acetate, cortodoxone, deflazacort, desonide, desoximetasone, dexamethasone, dexamethasone acetate, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

Alternatively, the anti-inflammatory agent can be a biological inhibitor of pro-inflammatory signaling molecules. Anti-inflammatory biological agents include antibodies to such biological inflammatory signaling molecules.

In addition, the bioactive agents can be other than antiproliferative or anti-inflammatory agents. The bioactive agents can be any agent that is a therapeutic, prophylactic or diagnostic agent. In some embodiments, such agents can be used in combination with antiproliferative or anti-inflammatory agents. These bioactive agents can also have antiproliferative and/or anti-inflammmatory properties or can have other properties such as antineoplastic, antimitotic, cystostatic, antiplatelet, anticoagulant, antifibrin, antithrombin, antibiotic, antiallergic, and/or antioxidant properties.

Examples of antineoplastics and/or antimitotics include, but are not limited to, paclitaxel (e.g., TAXOL® available from Bristol-Myers Squibb), docetaxel (e.g., Taxotere® from Aventis), methotrexate, azathioprine, vincristine, vinblastine, fluorouracil, doxorubicin hydrochloride (e.g., Adriamycin® from Pfizer), and mitomycin (e.g., Mutamycin® from Bristol-Myers Squibb).

Examples of antiplatelet, anticoagulant, antifibrin, and antithrombin agents that can also have cytostatic or antiproliferative properties include, but are not limited to, sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as ANGIOMAX (from Biogen), calcium channel blockers (e.g., nifedipine), colchicine, fibroblast growth factor (FGF) antagonists, fish oil (e.g., omega 3-fatty acid), histamine antagonists, lovastatin (a cholesterol-lowering drug that inhibits HMG-CoA reductase, brand name Mevacor® from Merck), monoclonal antibodies (e.g., those specific for platelet-derived growth factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetics, 4-amino-2,2,6,6-tetramethylpiperidine-1-oxyl(4-amino-TEMPO), estradiol, anticancer agents, dietary supplements such as various vitamins, and a combination thereof.

Examples of cytostatic substances include, but are not limited to, angiopeptin, angiotensin converting enzyme inhibitors such as captopril (e.g., Capoten® and Capozide® from Bristol-Myers Squibb), cilazapril and lisinopril (e.g., Prinivil® and Prinzide® from Merck).

Examples of antiallergic agents include, but are not limited to, permirolast potassium. Examples of antioxidant substances include, but are not limited to, 4-amino-2,2,6,6-tetramethylpiperidine-1-oxyl(4-amino-TEMPO). Other bioactive agents include anti-infectives such as antiviral agents; analgesics and analgesic combinations; anorexics; antihelmintics; antiarthritics, antiasthmatic agents; anticonvulsants; antidepressants; antidiuretic agents; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary vasodilators; peripheral and cerebral vasodilators; central nervous system stimulants; cough and cold preparations, including decongestants; hypnotics; immunosuppressives; muscle relaxants; parasympatholytics; psychostimulants; sedatives; tranquilizers; naturally derived or genetically engineered lipoproteins; and restenoic reducing agents.

Other biologically active agents that can be used include alpha-interferon, genetically engineered epithelial cells, tacrolimus and dexamethasone.

A “prohealing” drug or agent, in the context of a blood-contacting implantable device, refers to a drug or agent that has the property that it promotes or enhances re-endothelialization of arterial lumen to promote healing of the vascular tissue. The portion(s) of an implantable device (e.g., a stent) containing a prohealing drug or agent can attract, bind, and eventually become encapsulated by endothelial cells (e.g., endothelial progenitor cells). The attraction, binding, and encapsulation of the cells will reduce or prevent the formation of emboli or thrombi due to the loss of the mechanical properties that could occur if the stent was insufficiently encapsulated. The enhanced re-endothelialization can promote the endothelialization at a rate faster than the loss of mechanical properties of the stent.

The prohealing drug or agent can be dispersed in the body of the bioabsorbable polymer substrate or scaffolding. The prohealing drug or agent can also be dispersed within a bioabsorbable polymer coating over a surface of an implantable device (e.g., a stent).

“Endothelial progenitor cells” refer to primitive cells made in the bone marrow that can enter the bloodstream and go to areas of blood vessel injury to help repair the damage. Endothelial progenitor cells circulate in adult human peripheral blood and are mobilized from bone marrow by cytokines, growth factors, and ischemic conditions. Vascular injury is repaired by both angiogenesis and vasculogenesis mechanisms. Circulating endothelial progenitor cells contribute to repair of injured blood vessels mainly via a vasculogenesis mechanism.

In some embodiments, the prohealing drug or agent can be an endothelial cell (EDC)-binding agent. In certain embodiments, the EDC-binding agent can be a protein, peptide or antibody, which can be, e.g., one of collagen type 1, a 23 peptide fragment known as single chain Fv fragment (scFv A5), ajunction membrane protein vascular endothelial (VE)-cadherin, and combinations thereof. Collagen type 1, when bound to osteopontin, has been shown to promote adhesion of endothelial cells and modulate their viability by the down regulation of apoptotic pathways. S. M. Martin, et al., J. Biomed. Mater. Res., 70A: 10-19 (2004). Endothelial cells can be selectively targeted (for the targeted delivery of immunoliposomes) using scFv A5. T. Volkel, et al., Biochimica et Biophysica Acta, 1663:158-166 (2004). Junction membrane protein vascular endothelial (VE)-cadherin has been shown to bind to endothelial cells and down regulate apoptosis of the endothelial cells. R. Spagnuolo, et al., Blood, 103:3005-3012 (2004).

In a particular embodiment, the EDC-binding agent can be the active fragment of osteopontin, (Asp-Val-Asp-Val-Pro-Asp-Gly-Asp-Ser-Leu-Ala-Try-Gly). Other EDC-binding agents include, but are not limited to, EPC (epithelial cell) antibodies, RGD peptide sequences, RGD mimetics, and combinations thereof.

In further embodiments, the prohealing drug or agent can be a substance or agent that attracts and binds endothelial progenitor cells. Representative substances or agents that attract and bind endothelial progenitor cells include antibodies such as CD-34, CD-133 and vegf type 2 receptor. An agent that attracts and binds endothelial progenitor cells can include a polymer having nitric oxide donor groups.

The foregoing biologically active agents are listed by way of example and are not meant to be limiting. Other biologically active agents that are currently available or that may be developed in the future are equally applicable.

In a more specific embodiment, optionally in combination with one or more other embodiments described herein, the implantable device of the invention comprises at least one biologically active agent selected from paclitaxel, docetaxel, estradiol, nitric oxide donors, super oxide dismutases, super oxide dismutase mimics, 4-amino-2,2,6,6-tetramethylpiperidine-1-oxyl(4-amino-TEMPO), tacrolimus, dexamethasone, dexamethasone acetate, rapamycin, rapamycin derivatives, 40-O-(2-hydroxy)ethyl-rapamycin (everolimus), 40-O-(2-ethoxy)ethyl-rapamycin (biolimus), 40-O-(3-hydroxy)propyl-rapamycin, 40-O-[2-(2-hydroxy)ethoxy]ethyl-rapamycin, 40-O-tetrazole-rapamycin, 40-epi-(N1-tetrazolyl)-rapamycin (zotarolimus), Biolimus A9 (Biosensors International, Singapore), AP23572 (Ariad Pharmaceuticals), pimecrolimus, imatinib mesylate, midostaurin, clobetasol, progenitor cell-capturing antibodies, prohealing drugs, prodrugs thereof, co-drugs thereof, and a combination thereof. In a particular embodiment, the bioactive agent is everolimus. In another specific embodiment, the bioactive agent is clobetasol.

An alternative class of drugs would be p-para-α-agonists for increased lipid transportation, examples include feno fibrate.

In some embodiments, optionally in combination with one or more other embodiments described herein, the at least one biologically active agent specifically cannot be one or more of any of the bioactive drugs or agents described herein.

### Coating Construct

According to some embodiments of the invention, optionally in combination with one or more other embodiments described herein, a soluble coating can be disposed over an implantable device (e.g., a stent) in a layer according to any design of a coating. The coating can be a multi-layer structure that includes at least one reservoir layer, which is layer (2) described below, and can include any of the following (1), (3), (4) and (5) layers or combination thereof:

(1) a primer layer; (optional)

(2) a reservoir layer (also referred to “matrix layer” or “drug matrix”), which can be a drug-polymer layer including at least one polymer (drug-polymer layer) or, alternatively, a polymer-free drug layer;

(3) a release control layer (also referred to as a “rate-limiting layer”) (optional);

(4) a topcoat layer; and/or (optional);

(5) a finishing coat layer (optional).

In some embodiments, a coating of the invention can include two or more reservoir layers described above, each of which can include a bioactive agent described herein.

Each layer of a stent coating can be disposed over the implantable device (e.g., a stent) by dissolving the biosoluble polymer or copolymer, optionally with one or more other polymers, in a solvent, or a mixture of solvents, and disposing the resulting coating solution over the stent by spraying or immersing the stent in the solution. After the solution has been disposed over the stent, the coating is dried by allowing the solvent to evaporate. The process of drying can be accelerated if the drying is conducted at an elevated temperature. The complete stent coating can be optionally annealed at a temperature between about 40° C. and about 150° C., e.g., 80° C., for a period of time between about 5 minutes and about 60 minutes, if desired, to allow for crystallization of the polymer coating, and/or to improve the thermodynamic stability of the coating.

To incorporate a bioactive agent (e.g., a drug) into the reservoir layer, the drug can be combined with the polymer solution that is disposed over the implantable device as described above. Alternatively, if it is desirable a polymer-free reservoir can be made. To fabricate a polymer-free reservoir, the drug can be dissolved in a suitable solvent or mixture of solvents, and the resulting drug solution can be disposed over the implantable device (e.g., stent) by spraying or immersing the stent in the drug-containing solution.

Instead of introducing a drug via a solution, the drug can be introduced as a colloid system, such as a suspension in an appropriate solvent phase. To make the suspension, the drug can be dispersed in the solvent phase using conventional techniques used in colloid chemistry. Depending on a variety of factors, e.g., the nature of the drug, those having ordinary skill in the art can select the solvent to form the solvent phase of the suspension, as well as the quantity of the drug to be dispersed in the solvent phase. Optionally, a surfactant can be added to stabilize the suspension. The suspension can be mixed with a polymer solution and the mixture can be disposed over the stent as described above. Alternatively, the drug suspension can be disposed over the stent without being mixed with the polymer solution.

The drug-polymer layer can be applied directly or indirectly over at least a portion of the stent surface to serve as a reservoir for at least one bioactive agent (e.g., drug) that is incorporated into the reservoir layer. The optional primer layer can be applied between the stent and the reservoir to improve the adhesion of the drug-polymer layer to the stent. The optional topcoat layer can be applied over at least a portion of the reservoir layer and serves as a rate-limiting membrane that helps to control the rate of release of the drug. In one embodiment, the topcoat layer can be essentially free from any bioactive agents or drugs. If the topcoat layer is used, the optional finishing coat layer can be applied over at least a portion of the topcoat layer for further control of the drug-release rate and for improving the biocompatibility of the coating. Without the topcoat layer, the finishing coat layer can be deposited directly on the reservoir layer.

Sterilization of a coated medical device generally involves a process for inactivation of micropathogens. Such processes are well known in the art. A few examples are e-beam, ETO sterilization, and irradiation. Most, if not all, of these processes can involve an elevated temperature. For example, ETO sterilization of a coated stent generally involves heating above 50° C. at humidity levels reaching up to 100% for periods of a few hours up to 24 hours. A typical EtO cycle would have the temperature in the enclosed chamber to reach as high as above 50° C. within the first 3-4 hours then and fluctuate between 40° C. to 50° C. for 17-18 hours while the humidity would reach the peak at 100% and maintain above 80% during the fluctuation time of the cycle.

The process of the release of a drug from a coating having both topcoat and finishing coat layers includes at least three steps. First, the drug is absorbed by the polymer of the topcoat layer at the drug-polymer layer/topcoat layer interface. Next, the drug diffuses through the topcoat layer using the void volume between the macromolecules of the topcoat layer polymer as pathways for migration. Next, the drug arrives at the topcoat layer/finishing layer interface. Finally, the drug diffuses through the finishing coat layer in a similar fashion, arrives at the outer surface of the finishing coat layer, and desorbs from the outer surface. At this point, the drug is released into the blood vessel or surrounding tissue. Consequently, a combination of the topcoat and finishing coat layers, if used, can serve as a rate-limiting barrier. The drug can be released by virtue of the degradation, dissolution, and/or erosion of the layer(s) forming the coating, or via migration of the drug through the polymeric layer(s) into a blood vessel or tissue.

In one embodiment, any or all of the layers of the soluble stent coating can be made of a polymer or copolymer described herein. In another embodiment, the outermost layer of the coating can be limited to a polymer or copolymer as defined above.

To illustrate in more detail, in a soluble stent coating having all four layers described above (i.e., the primer, the reservoir layer, the topcoat layer and the finishing coat layer), the outermost layer is the finishing coat layer, which can be made of a polymer or copolymer described. The remaining layers (i.e., the primer, the reservoir layer and the topcoat layer) optionally having the properties of being soluble and biodegradable or, biostable, or being mixed with a polymer or copolymer as described herein. The polymer(s) in a particular layer may be the same as or different than those in any of the other layers, as long as the layer on the outside of another soluble polymer should preferably also dissolve at a similar or faster relative to the inner layer. As another illustration, the coating can include a single matrix layer comprising a polymer described herein and a drug.

If a finishing coat layer is not used, the topcoat layer can be the outermost layer and should be made of a polymer or copolymer as described. In this case, the remaining layers (i.e., the primer and the reservoir layer) optionally can also be fabricated of a polymer or copolymer described herein. The polymer(s) in a particular layer may be the same as or different than those in any of the other layers, as long as the outside of another soluble polymer should preferably also dissolve at a similar or faster relative to the inner layer.

If neither a finishing coat layer nor a topcoat layer is used, the stent coating could have only two layers—the primer and the reservoir. In such a case, the reservoir is the outermost layer of the stent coating and should be made of a polymer or copolymer described. The primer optionally can also be fabricated of a polymer or copolymer described herein and optionally one or more soluble biodegradable polymer(s), biostable polymer(s), or a combination thereof. The two layers may be made from the same or different polymers, as long as the layer on the outside of another soluble polymer should preferably also dissolve at a similar or faster relative to the inner layer.

Any layer of a coating can contain any amount of a polymer or copolymer described herein and optionally being mixed with another soluble bioabsorbable and/or biocompatible polymer.

Any layer of a coating can also contain any amount of a soluble, non-degradable polymer, or a blend of more than one such. When a non-degradable polymer is used, the non-degradable polymer shall have a molecular weight (M_{w}) of about 40K Daltons or below. In general since not many polymers are very elastic enough. If they are not, the higher Mw will provide the toughness for the coating, ideally in drug eluting stents, polymers of higher than 100 kD are preferable. Also, since they are not degradable or soluble, clearing the kidney should not be a concern. Non-limiting examples of soluble non-degradable polymers include poly(2-hydroxylethyl methacrylate), poly(ethylene glycol (PEG) acrylate), poly(PEG methacrylate), methacrylate polymers containing 2-methacryloyloxyethylphosphorylcholine (MPC), PC1036, and poly(n-vinyl pyrrolidone, poly(hydroxypropyl methacrylamide), soluble methacrylamide polymers, or soluble acrylarnide polymers, and copolymers thereof.

In some embodiments, the soluble coating described herein can specifically exclude any of the above listed polymers.

### Method of Treating or Preventing Disorders

An implantable device according to the present invention can be used to treat, prevent or diagnose various conditions or disorders. Examples of such conditions or disorders include, but are not limited to, atherosclerosis, thrombosis, restenosis, hemorrhage, vascular dissection, vascular perforation, vascular aneurysm, vulnerable plaque, chronic total occlusion, patent foramen ovale, claudication, anastomotic proliferation of vein and artificial grafts, arteriovenous anastamoses, bile duct obstruction, urethral obstruction and tumor obstruction. A portion of the implantable device or the whole device itself can be formed of the material, as described herein. For example, the material can be a coating disposed over at least a portion of the device.

In certain embodiments, optionally in combination with one or more other embodiments described herein, the inventive method treats, prevents or diagnoses a condition or disorder selected from atherosclerosis, thrombosis, restenosis, hemorrhage, vascular dissection, vascular perforation, vascular aneurysm, vulnerable plaque, chronic total occlusion, patent foramen ovale, claudication, anastomotic proliferation of vein and artificial grafts, arteriovenous anastamoses, bile duct obstruction, urethral obstruction and tumor obstruction. In a particular embodiment, the condition or disorder is atherosclerosis, thrombosis, restenosis or vulnerable plaque.

In certain embodiments, optionally in combination with one or more other embodiments described herein, the implantable device used in the method is selected from stents, grafts, stent-grafts, catheters, leads and electrodes, clips, shunts, closure devices, valves, and particles. In a specific embodiment, the implantable device is a stent.

### EXAMPLE

The following non-limiting example shows a biosoluble coating with linear over time mass loss:

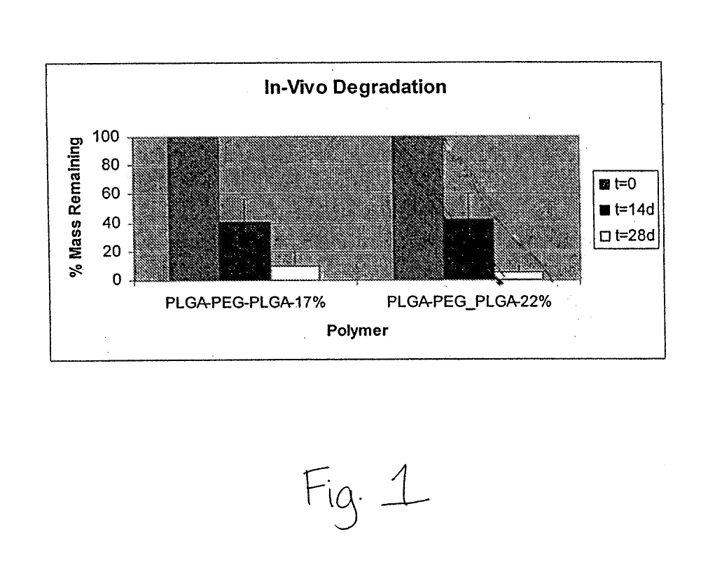

Coatings were formed of PDLGA-PEG-PDLGA block copolymer on Vision stents (available from Abbott Cardiovascular, Santa Clara, Calif.) with 70:30 ratio of PDLA:PGA and having PEG compositions of 17 mass % and 22 mass % showed greater than 80 mass % and 90 mass % mass loss respectively for polymers containing 17 mass % and 22 mass % PEG at 28 days using the in-vivo porcine model (FIG. 1). Additionally, as seen in FIG. 1, linear mass loss was observed from t=0 to t=28 days. This linear mass loss is indicative of surface erosion for the tested biosoluble coatings. In these studies, formulation with drug-to-polymer ratio of 1 to 3 was used for both polymers. Both formulations showed acceptable coating integrity after expansion and simulated use at 1 hour and 24 hours (Data not shown).

While particular embodiments of the present invention have been shown and described, it will be obvious to those skilled in the art that changes and modifications can be made without departing from this invention in its broader aspects. Therefore, the claims are to encompass within their scope all such changes and modifications as fall within the true sprit and scope of this invention.

1. An implantable device, comprising a coating that dissolves or absorbs upon exposure to a physiological environment with linear over time mass loss (LOTML) at a rate such that about 80% or more of coating mass dissolves or absorbs within about 30 days after implantation of the implantable device.

2. The implantable device of claim 1, wherein the coating comprises a polymer selected from polyethylene glycol (PEG), copolymers comprising PEG, polyvinyl alcohol (PVA), poly(vinyl pyrrolidone) (PVP), hyaluronic acid, hydroxyl cellulose, polysaccharides, phosphoryl choline polymers, or combinations thereof.

3. The implantable device of claim 1, wherein the coating comprises a poly(lactic acid-co-glycolic acid)-co-polyethylene glycol) (PLGA-PEG) block copolymer.

4. The implantable device of claim 1, wherein the coating comprises a hydrophilic polymer.

5. The implantable device of claim 1, wherein the coating comprises a hydrophobic component.

6. The implantable device of claim 5, wherein the hydrophobic component comprises a hydrophobic polymer.

7. The implantable device of claim 1, wherein the biosoluble coating comprises PDLGA-PEG-PDLGA block copolymer.

8. The implantable device of claim 7, wherein the PDLGA-PEG-PDLGA block copolymer comprises a PEG content ranging from about 5 mass % to about 50 mass %.

9. The implantable device of claim 7, wherein the PDLGA-PEG-PDLGA block copolymer comprises a PEG content ranging from about 15 mass % to about 30 mass %.

10. The implantable device of claim 1, wherein the coating further comprises a bioactive agent.

11. The implantable device of claim 10, wherein the bioactive agent is selected from paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl(4-amino-TEMPO), biolimus, tacrolimus, dexamethasone, dexamethasone acetate, corticosteroids, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), zotarolimus, Biolimus A9 (Biosensors International, Singapore), AP23572 (Ariad Pharmaceuticals), γ-hiridun, clobetasol, pimecrolimus, imatinib mesylate, midostaurin, cRGD, feno fibrate, peptides, prodrugs thereof, co-drugs thereof, and combinations thereof.

12. The implantable device of claim 1, which is a stent.

13. The implantable device of claim 1, which is a bioabsorbable stent.

14. The implantable device of claim 10, which is a stent.

15. The implantable device of claim 11, which is a stent.

16. A method of fabricating an implantable device, comprising forming a coating that dissolves or absorbs upon exposure to a physiological environment with linear over time mass loss (LOTML) at a rate such that about 80% or more of coating mass dissolves or absorbs within about 30 days after implantation of the implantable device.

17. The method of claim 16, wherein the coating comprises a polymer selected from polyethylene glycol (PEG), copolymers comprising PEG, polyvinyl alcohol (PVA), poly(vinyl pyrrolidone) (PVP), hyaluronic acid, hydroxyl cellulose, polysaccharides, phosphoryl choline polymers, or combinations thereof.

18. The method of claim 16, wherein the coating comprises a poly(lactic acid-co-glycolic acid)-co-polyethylene glycol) (PLGA-PEG) block copolymer.

19. The method of claim 6, wherein the coating comprises a hydrophilic polymer.

20. The method of claim 16, wherein the coating comprises a hydrophobic component.

21. The method of claim 20, wherein the hydrophobic component comprises a hydrophobic polymer.

22. The method of claim 16, wherein the biosoluble coating comprises PDLGA-PEG-PDLGA block copolymer.

23. The method of claim 22, wherein the PDLGA-PEG-PDLGA block copolymer comprises a PEG content ranging from about 5 mass % to about 50 mass %.

24. The method of claim 22, wherein the PDLGA-PEG-PDLGA block copolymer comprises a PEG content ranging from about 15 mass % to about 30 mass %.

25. The method of claim 16, wherein the coating further comprises a bioactive agent.

26. The method of claim 25, wherein the bioactive agent is selected from paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl(4-amino-TEMPO), biolimus, tacrolimus, dexamethasone, dexamethasone acetate, corticosteroids, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), zotarolimus, Biolimus A9 (Biosensors International, Singapore), AP23572 (Ariad Pharmaceuticals), γ-hiridun, clobetasol, pimecrolimus, imatinib mesylate, midostaurin, cRGD, feno fibrate, peptides, prodrugs thereof, co-drugs thereof, and combinations thereof.

27. The method of claim 16, wherein the implantable device is a stent.

28. The method of claim 16, wherein the implantable device is a bioabsorbable stent.

29. The method of claim 25, wherein the implantable device is a stent.

30. The method of claim 26, wherein the implantable device is a stent.

31. A method, comprising implanting in a human being an implantable device according to claim 1 for treating, preventing or ameliorating a medical 5 condition selected from the group consisting of restenosis, atherosclerosis, thrombosis, hemorrhage, vascular dissection or perforation, vascular aneurysm, vulnerable plaque, chronic total occlusion, claudication, anastomotic proliferation (for vein and artificial grafts), bile duct obstruction, urethral obstruction, tumor obstruction, or combinations of these.

32. A method, comprising implanting in a human being an implantable device according to claim 11 for treating, preventing or ameliorating a medical condition selected from the group consisting of restenosis, atherosclerosis, thrombosis, hemorrhage, vascular dissection or perforation, vascular aneurysm, vulnerable plaque, chronic total occlusion, claudication, anastomotic proliferation (for vein and artificial grafts), bile duct obstruction, urethral obstruction, tumor obstruction, or combinations of these.
